(19) United States
(12) Patent Application Publication
(10) Pub. No.: US 2010/0009451 A1
(43) Pub. Date: Jan. 14, 2010

(54) COMPOSITIONS AND METHODS FOR SPECIFICALLY SILENCING A TARGET NUCLEIC ACID

(75) Inventors: Erik R. Eastlund, Fenton, MO (US); Greg D. Davis, Webster Groves, MO (US); Derek K. Douglas, St. Louis, MO (US); David K. Stone, Omaha, NE (US)

Correspondence Address: POLSINELLI SHUGHART PC, 700 W. 47TH STREET, SUITE 1000, KANSAS CITY, MO, 64112-1802, US

(73) Assignee: SIGMA ALDRICH COMPANY, St. Louis, MO (US)

(21) Appl. No.: 12/474,395

(22) Filed: May 29, 2009

Related U.S. Application Data
(60) Provisional application No. 61/057,270, filed on May 30, 2008.

(51) Int. Cl. C12N 15/87; C07H 21/04

(52) U.S. Cl. 435/463; 536/24.5

(57) ABSTRACT

The present invention provides methods modified oligonucleotides and methods of using the modified oligonucleotides for silencing nucleic acids, wherein the nonspecific effects of nucleic acid silencing are reduced.

### CROSS REFERENCE TO RELATED APPLICATIONS

This application claims the priority of U.S. provisional application No. 61/057,270, filed May 30, 2008, which is hereby incorporated by reference in its entirety.

### FIELD OF THE INVENTION

The present invention generally relates to the silencing of nucleic acids by small interfering RNAs. In particular, it relates to modified oligonucleotides and methods of using the modified oligonucleotides for silencing nucleic acids, wherein the nonspecific effects of nucleic acid silencing are reduced.

### BACKGROUND OF THE INVENTION

RNA interference or RNA silencing is a natural process that reduces the expression of specific messenger RNAs (mRNAs). RNA interference is mediated by small interfering RNAs (siRNAs). Upon incorporation of the antisense (or guide) strand of the siRNA duplex into the RNA-induced silencing complex (RISC), the antisense strand base pairs with a complementary target, which is then silenced by degradation and/or inhibition of translation. While synthetic siRNAs are able to silence specific targets, they may also silence unintended targets. This nonspecific silencing is termed siRNA off-targeting. Off-targeting may be mediated by the sense strand (i.e., it may erroneously enter RISC) or it may be mediated by a small region of the antisense strand (i.e., the seed region) that binds to complementary seed matches in other transcripts.

A variety of approaches have been undertaken to reduce or eliminate siRNA off-targeting. For example, chemical modifications in certain residues of siRNA duplexes have been shown to reduce, but not eliminate, off-target effects. There is a need, therefore, for improved methods for minimizing siRNA off-targeting and increasing siRNA specificity.

### SUMMARY OF THE INVENTION

Among the various aspects of the present invention is the provision of method for specifically silencing the expression of a target nucleic acid in a biological sample by RNA interference. In particular, the method comprises contacting the biological sample with an oligonucleotide comprising a duplex portion, wherein the duplex portion comprises a sense region base paired with an antisense region. Additionally, the antisense region of the duplex portion of the oligonucleotide has at least about 70% complementary to the target nucleic acid, and the antisense region also comprises at least one 2′-5′ internucleotide linkage in the region from the second nucleotide to the eighth nucleotide from the 5′ end.

A further aspect of the invention encompasses an oligonucleotide comprising a duplex portion comprising a sense region base paired with an antisense region. Furthermore, the antisense region comprises a 5′ phosphate group on the first nucleotide and at least one 2′-5′ internucleotide linkage in the region from the second nucleotide to the eighth nucleotide from the 5′ end.

Other aspects and features of the invention are described in more detail below.

### DESCRIPTION OF THE FIGURES

FIG. 1 presents the percent of expression of target, off-target, and control nucleic acids after exposure to modified or unmodified MAPK14 siRNAs. The off-target nucleic acids were ANKFY1, CTNNB1, and MARK2, and the control nucleic acid was CSNK1A1. The MAPK14 siRNAs were unmodified (MAPK14-193) or modified with 2′-O-methyl, 2′-methoxyethoxy, 2′-allyl, or 2′-5-linkage modifications.

FIG. 2 illustrates the percent of expression of MAPK14 after exposure to MAPK14 siRNAs having a different sequence than that used in FIG. 1. The siRNA was unmodified (MAPK14-6 normal) or modified with 2′-O-methyl, 2′-methoxyethoxy, 2′-allyl, 2′-5-linkage, 2′ amino, or 2′-dimethylallyl substituents.

FIG. 3 presents the expression of target, off-target, and control nucleic acids in a microarray analysis. Plotted is the intensity of the expression signal (±SEM) in mock treated samples or samples treated with modified or unmodified MAPK14 siRNAs. The MAPK14 siRNAs were unmodified (193) or modified with a 2′-O-methyl substituent or a 2′-5-linkage. (A) Presents a plot of the intensity of expression of the target MAPK14 as a function of siRNA. (B) Presents a plot of the expression of the off-target CTNNB1 for each of the siRNAs. (C) Presents a plot of the expression of the off-target ANKFY1 as a function of siRNA. (D) Presents a plot of the expression of the off-target MARK2 for each of the siRNAs. (E) Presents a plot of the expression of the control CSNK1A1 as a function of siRNA.

FIG. 4 illustrates the off-target reduction ratio of the 2′-5′-linked to the 2′-O-methyl MAPK14 siRNAs at different intensity cut off levels and intensity threshold levels. (A) Presents the ratio for the MAPK14-193 siRNAs. (B) Presents the ratio for the MAPK14-6 siRNAs.

FIG. 5 presents the number of remaining off-targets after exposure to unmodified or 2′-O-methyl or 2′-5′-linked MAPK14 siRNAs. (A) Presents a plot of the number of remaining off-targets for the MAPK14-193 siRNAs. (B) Presents a plot of the number of remaining off-targets for the MAK14-6 siRNAs.

FIG. 6 depicts the number of potential off-targets remaining after exposure to either unmodified or 2′-5′-linked (modified) PPP2R2A siRNAs. Plotted is the number of potential off-targets remaining for each siRNA at different intensity levels. The p-cutoff was 0.01.

FIG. 7 illustrates the lowest effective siRNA concentration for normal (i.e., unmodified) and modified (i.e., 2′-5′-linkage) siRNAs in global off-target reduction. (A) Presents a plot of the percent knockdown of TP53 as a function of siRNA concentration. (B) Presents a plot of the number of potential off-targets remaining for each siRNA at different intensity levels. The p-cutoff was 0.01.

FIG. 8 presents the effects of scrambled negative control siRNA on global siRNA off-target reduction. (A) Presents a plot of the number of potential off-targets remaining for normal (i.e., unmodified) and modified (i.e., 2′-5′-linkage) negative control sequence 12. (B) Presents a plot of the number of potential off-targets remaining for the normal (i.e., unmodified) and modified (i.e., 2′-5′-linkage) negative control sequence 13. The p-cutoff for each was 0.0001.

FIG. 9 illustrates specific knockdowns using either unmodified or modified (2′-5′-linked) siRNAs. The percent of gene expression is plotted for each type of RNA for 24 different genes.

FIG. 10 presents a comparison of global off-target reduction using different passenger strand designs. (A) Presents a plot of the intensity of expression for each type of siRNA. (B) Presents a plot of the potential off-targets remaining for each of the siRNAs as a function of off-target reduction thresholds. The p-cutoff was 0.01.

### DETAILED DESCRIPTION

The present invention provides a method for specifically silencing a target nucleic acid, as well as an oligonucleotide for use in the method. The silencing of the target nucleic acid is mediated by RNA interference. The method utilizes an oligonucleotide comprising a duplexed sense and antisense portion, wherein the antisense region comprises at least one 2′-5′ internucleotide linkage in the seed region (i.e., the region encompassing the second to the eighth nucleotide from the 5′ end). It has been discovered that oligonucleotides comprising a 2′-5′ internucleotide linkage in the seed region have reduced off-target effects relative to other siRNAs having other chemical modifications.

### (I) Method for Specific Silencing a Target Nucleic Acid

One aspect of the present invention provides a method for specifically silencing a target nucleic acid in a biological sample. The method comprises contacting the biological sample with an oligonucleotide comprising a duplex portion. The duplex portion of the oligonucleotide comprises a sense region that is base paired with an antisense region. The antisense region of the oligonucleotide has at least about 70% complementary to the target nucleic acid, and the antisense region comprises at least one 2′-5′ internucleotide linkage in the region from the second nucleotide to the eighth nucleotide from the 5′ end.

### (a) Oligonucleotide

The composition and structure of the oligonucleotide can and will vary. The oligonucleotide comprises a plurality of linked nucleotides, and the moieties of the nucleotides, the type of linkages between the nucleotides, as well as the structure of the oligonucleotide may vary.

### (i) Nucleotides

The nucleotides comprising the oligonucleotide may be ribonucleotides, deoxynucleotides, deoxyribonucleotides, derivatized nucleotides, modified nucleotides, nucleotide analogs, or combinations thereof. In general, a deoxynucleotide refers to a nucleotide that does not have a hydroxyl group attached to the 2′ carbon or the 3′ carbon of the sugar moiety of the nucleotide; and a deoxyribonucleotide refers to a nucleotide that does not have a hydroxyl group attached to the 2′ carbon of the sugar moiety.

The sugar moiety of the nucleotide may be an acyclic sugar or a carbocyclic sugar. Suitable examples of an acyclic sugar include, but are not limited to glycerol (which may form a glycerol nucleic acid or GNA), threose (which may form a threose nucleic acid or TNA), erthrulose, erythrose, and so forth. Non-limiting examples of suitable carbocyclic sugars include pentoses (such as, arabinose, deoxyribose, lyxose, ribose, xylose, xylulose, etc., and derivatives thereof) and hexoses (such as, galactose, glucose, mannose, etc., and derivatives thereof). The sugar moiety may be isomeric, i.e., it may be the D-form or the L-form. The configuration of the sugar moiety may be alpha (α) or beta (β). The sugar moiety of a nucleotide also may comprise a locked nucleic acid (LNA), in which the 2′ and 4′ carbons, or the 3′ and 4′ carbons, of the sugar moiety are connected with an extra bridge. The nucleotide may also comprise a sugar analog or substitute, such as a morpholine ring, which may be connected by a phorphorodiamidate linkage to form a morpholino, or a N-(2-aminoethyl)-glycine unit, which may be connected by a peptide bond to form a peptide nucleic acid (PNA). In preferred embodiments, the sugar moiety may be a β-D-ribose.

The sugar moiety of the nucleotide also may have a substituent at the 2′ position or the 3′ position of the molecule. The substituent may be selected from the group consisting of hydrogen, halogen, —R, —NHR, —NRR¹, —SR, and —OR, wherein R and R¹ are independently selected from the group consisting of hydrogen, hydrocarbyl, and substituted hydrocarbyl. Preferably, R may be alkyl (such as, e.g., methyl, ethyl, propyl, isopropyl, etc), acyl, alkenyl, or aryl. In preferred embodiments, the substituent may be fluoro, amino, methyl, —O-alkyl, or —O-acyl. In an exemplary embodiment, the substituent may be —O-methyl.

The heterocyclic base moiety of the nucleotide may be an unmodified purine base (e.g, adenine, guanine, hypoxanthine, or xanthine) or an unmodified prymidine base (e.g., cytosine, thymine, or uracil). Alternatively, the purine or pyrimidine base moiety may be a derivatized or modified by the replacement or addition of one of more atoms or groups. Examples of suitable modifications include, but are not limited to, alkylation, halogenation, thiolation, amination, amidation, acetylation, and combinations thereof. More specific modified bases include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. In preferred embodiments, the base moiety may be a standard purine or pyrimidine (i.e., adenine, cytosine, guanine, thymine, and uracil) base.

Furthermore, one or more of the functional groups of a base moiety may be protected with a protecting group. Examples of suitable protecting groups are well known in the art. The base moiety may also be conjugated to a marker molecule such as a fluorophore, biotin, digoxigenin, or other such molecule that is known in the art.

### (ii) Internucleotide Linkage

The nucleotides of the oligonucleotide may be connected by phosphorus-containing linkages, non-phosphorus-containing linkages, or combinations thereof. Examples of suitable phosphorus-containing linkages include, but are not limited to, phosphodiester, phosphorothioate, phosphorodithioate, phosphoramidate, alkylphosphoramidate, aminoalkylphosphoramidate, thionophosphoramidate, alkylphosphonothioate, arylphosphonothioate, thiophosphate, alkyl phosphonate, methylphosphonate, alkylenephosphonate, hydrogen phosphonate, phosphotriester, ethylphosphotriester, thionoalkylphosphotriester, phosphinate, borano phosphate ester, selenophosphate, phosphoroselenoate, phosphorodiselenoate, phosphoropiperazidate, phosphoroanilothioate, and phosphoroanilidate linkages. Non-limiting examples of suitable non-phosphorus-containing linkages include alkyl, amide, amine, aminoethyl glycine, borontrifluoridate, carbamate, carbonate, cycloalkyl, ether, formacetal, glycol, hydroxylamine, hydrazino, ketone, methylenehydrazo, methylenedimethylhydrazo, methyleneimino, methylene(methylimino), methylester, oxime, sulfonamide, sulfone, thioamidate, siloxane, silyl, thioformacetal, and urea linkages. In preferred embodiments, the internucleotide linkages may be phosphodiester or phosphorothioate linkages. In an exemplary embodiment, the internucleotide linkages may be phosphodiester linkages.

The oligonucleotide comprises at least one 2′-5′ linkage between the 2^{nd} and the 8^{th} nucleotides from the 5′ end of the antisense region (i.e., the seed region). Accordingly, the rest of the internucleotide linkages of the oligonucleotide may be either 3′-5′ or 2′-5′. Furthermore, the number of 2′-5′ linkages within the oligonucleotide can and will vary. In one embodiment, the oligonucleotide may comprise one, two, three, four, five, or six 2′-5′ linkages in the seed region, with the rest of the internucleotide linkages of the oligonucleotide being either 3′-5′ or 2′-5′. In another embodiment, the oligonucleotide may comprise one, two, three, four, five, or six 2′-5′ linkages in the seed region, at least one 2′-5′ linkage in the sense region of the oligonucleotide, with the rest of the internucleotide linkages of the oligonucleotide being either 3′-5′ or 2′-5′. In an exemplary embodiment, the oligonucleotide may comprise a 2′-5′ linkage between the 2^{nd} and 3^{rd} nucleotides from the 5′ end of the antisense region, with the rest of the internucleotide linkages being 3′-5′. In another exemplary embodiment, the oligonucleotide may comprise a 2′-5′ linkage between the 2^{nd} and 3^{rd} nucleotides from the 5′ end of the antisense region, a 2′-5′ linkage between the 2^{nd} and 3^{rd} nucleotides from the 5′ end of the sense region, with the rest of the internucleotide linkages being 3′-5′.

The oligonucleotides of the invention may be synthesized according to standard techniques using phorphoramidite monomers (e.g., Methods in Molecular Biology, Vol 20, Protocols for Oligonucleotides and Analogs, Agrawal, ed., Humana Press, Totowa, N.J., 1993). When a 2′-5′ linkage is desired, a suitably 3′ protected nucleotide monomer (such as a 3′-t-butylmethylsilyl-2′-beta-cyanoethyl phosphoramidite monomer) is typically used at the appropriate point in the stepwise synthesis.

### (iii) Oligonucleotide Structure

The duplex portion of the oligonucleotide comprises a sense region that is base paired with an antisense region. In general, the sense region and the antisense region of the oligonucleotide will have at least about 50% complementarity between such that they may base pair and form a duplex. Thus, the sense and antisense regions of the oligonucleotide may have about 50%, about 60%, about 70%, about 80%, about 90%, or about 100% complementarity.

In general, the length of the duplex portion of the oligonucleotide may range from about 15 base pairs to about 40 base pairs. In one embodiment, the duplex portion of the oligonucleotide may range from about 15 base pairs to about 20 base pairs. In another embodiment, the duplex portion of the oligonucleotide may range from about 20 base pairs to about 25 base pairs. In still another embodiment, the duplex portion of the oligonucleotide may range from about 25 base pairs to about 30 base pairs. In a further embodiment, the duplex portion of the oligonucleotide may range from about 30 base pairs to about 40 base pairs. In preferred embodiments, the duplex portion of the oligonucleotide may range from about 17 base pairs to about 25 base pairs.

In general, the antisense region will have at least about 70% complementarity to the target nucleic acid. Thus, the antisense region may have about 70%, about 75%, about 80%, about 85%, about 90%, about 95%, or about 100% complementarity to the target nucleic acid. Stated another way, if the antisense region is about 20 nucleotides in length, there may be about 6, 5, 4, 3, 2, 1, or zero mismatches (with respect to the target nucleic acid). Similarly, if the antisense region is about 25 nucleotides in length, there may be about 7, 6, 5, 4, 3, 2, 1, or zero mismatches (with respect to the target nucleic acid), and so forth. In a preferred embodiment, the antisense region may be the exact complement of a region of the target nucleic acid.

Generally, the antisense region will have complementary to a region of the target nucleic acid with low GC content and no predictable secondary structure. The antisense region may be designed using commercially available programs or services (e.g., Rosetta siRNA Design Algorithm from Sigma-Aldrich, St. Louis, Mo.; SILENCER® siRNA Design Algorithm from Ambion, Austin, Tex.; HiPerformance siRNA Design Algorithm from Qiagen, Valencia, Calif.; SMARTSELECTION™ siRNA Design Algorithm from Dharmacon, Lafayette, Colo.), public on-line services (e.g., Henschel et al. 2004, Nucl. Acid Res. 32:W113-120), or open-source programs (e.g., Holen, 2006, RNA 12:1620-1625).

In general, the oligonucleotide of the invention will comprise at least one strand of linked nucleotides. In one embodiment, the oligonucleotide may be a double-stranded molecule comprising one sense strand and one antisense strand, wherein the sense strand essentially comprises the sense region and the antisense strand essentially comprises the antisense region of the duplex portion. The oligonucleotide may comprise at least one 3′ overhang, i.e., a single-stranded region that extends beyond the duplex portion of the molecule. For example, the 3′ end of the sense strand, the 3′ end of the antisense strand, or both may extend beyond the duplex portion of the molecule. The 3′ overhang may range from about one nucleotide to about six nucleotides, or more preferably, from about one nucleotide to about three nucleotides. The 5′ terminal nucleotides of the sense and antisense strands of the oligonucleotide may also comprise substituents. For example, the first nucleotide at the 5′ end of the antisense strand may comprise one or more phosphate groups or phosphate group analogs. In a preferred embodiment, the first nucleotide at the 5′ end of the antisense strand may comprise one phosphate group. In other embodiments, the first nucleotide at the 5′ end of the sense strand may comprise an amino group. The amino group may be directly attached to the oxygen function at the 5′ carbon, it may be attached via a 5′ terminal phosphate group, or it may be attached via an alkyl or alkenyl linker to either of the above.

In another embodiment, the oligonucleotide may comprise two or more sense strands, as well as an antisense strand (Bramsen et al. 2007, Nucl. Acids Res. 35(17):5886-5897). The two or more sense strands generally base pair with the antisense strand. The two or more sense strands that are base paired with the antisense strand may be separated by a nick (i.e., there is no internucleotide bond between the terminal nucleotides of two adjacent sense strands). Alternatively, the two or more sense strands that are base paired with the antisense strand may be separated by a gap of one to two nucleotides. The oligonucleotides of this embodiment may also comprise at least one 3′ overhang as detailed above. Additionally, the first nucleotide at the 5′ end of the antisense strand may bear one or more phosphate group or phosphate group analogs, and the first nucleotide at the 5′ end of the sense strand may bear an amino group as detailed above.

In a further embodiment, the oligonucleotide may be a single stranded molecule comprising the duplex portion and a loop region, wherein the loop region connects the duplexed sense and antisense regions. The loop region may form a hairpin loop, a short hairpin loop, a bubble loop, or another loop structure. The length of the loop region may range from about 3 nucleotides to about 100 nucleotides, or preferably from about 20 nucleotides to about 35 nucleotides. The antisense region typically will be located at the 5′ end of the single-stranded molecule, and there may be a 3′ overhang at the other end of the molecule.

The length of the oligonucleotide can and will vary, depending upon the embodiment. In embodiments in which the oligonucleotide comprises a single strand, the oligonucleotide may range from about 33 nucleotides to about 180 nucleotides, or more preferably, from about 55 nucleotides to about 85 nucleotides. In embodiments in which the oligonucleotide comprises two or more strands, the length of the duplex portion of the oligonucleotide may range from about 15 base pairs to about 40 base pairs (not including single-stranded 3′ overhangs).

### (iv) Preferred Embodiments

In preferred embodiments, the oligonucleotide may comprise one sense and one antisense strand, wherein the length of the duplexed portion of the molecules may be about from about 19 to 21 base pairs, with 3′ overhangs of about 2 nucleotides. In one exemplary embodiment, the oligonucleotide may comprise a 2′-5′ internucleotide linkage between the second and third nucleotides from the 5′ end of the antisense strand, there may be a 5′ phosphate group on the first nucleotide from the 5′ end the antisense strand, and there may be a 5′ amino group on the first nucleotide from the 5′ end of the sense strand. In another exemplary embodiment, the oligonucleotide may comprise a 2′-5′ internucleotide linkage between the second and third nucleotides from the 5′ end of the antisense strand, there may be a 5′ phosphate group on the first nucleotide from the 5′ end the antisense strand, and there may be a 2′-O-methyl group on each of the first and second nucleotides from the 5′ end of the sense strand. In still another exemplary embodiment, oligonucleotide may comprise a 2′-5′ internucleotide linkage between the second and third nucleotides from the 5′ end of the antisense strand, there may be a 5′ phosphate group on the first nucleotide from the 5′ end the antisense strand, there may be a 2′-O-methyl group on the first nucleotide from the 5′ end the sense strand comprises, and there may be a 2′-5′ linkage between the second and third nucleotides from the 5′ end of the sense strand.

### (b) Biological Sample

The method of the invention comprises contacting the biological sample comprising the target nucleic acid with the oligonucleotide of the invention. The biological sample may be a cell or an extract of a cell. The cell may be a microbial or a fungal cell, a plant cell, or it may be derived from a multicellular animal. Suitable examples of a multicellular animals include invertebrates (e.g., Drosophila species) and vertebrates (e.g., frogs, zebrafish, rodents, and mammals such as companion animals, zoo animals, and humans). The cell may be in vitro (e.g., primary cell, cultured cell, or immortal cell line) or the cell may be in vivo.

Delivery of the oligonucleotide into the cell may be achieved by liposomal or other vesicular delivery systems, electroporation, direct cell fusion, viral carriers, osmotic shock, application of protein carriers or antibody carriers, and calcium-phosphate mediated transfection. To facilitate entry into the cell, the oligonucleotide may be chemically modified to enhance its permeability. Examples of receptor mediated endocytotic systems whereupon chemical conjugation to the oligonucleotide may be used to enhance cellular uptake by targeting a specific cell surface receptor include, but are not limited to, galactose, mannose, mannose-6-phosphate, transferrin, asialoglycoproteins, water soluble vitamins (e.g. transcobolamin, biotin, ascorbic acid, folates, etc.) any pharmacological agent or analog that mimics the binding of a water soluble vitamin, alpha-2 macroglobulins, insulin, epidermal growth factor, or attachment to an antibody against a surface protein of the target cell as in the case of the so-called immunotoxins. Chemical conjugation of the oligonucleotide may also include apolar substituents such as hydrocarbon chains or aromatic groups and/or polar substituents such as polyamines to further enhance intracellular uptake. Chemical conjugation of the oligonucleotide to an exogenous molecule may be achieved by covalent, ionic or hydrogen bonding either directly or indirectly by a linking group. Preferably, the exogenous molecule may be covalently linked to the oligonucleotide using techniques are well known in the art.

Various methods of formulation and administration of the oligonucleotide are known to those skilled in the medical arts (Avis, K. in Remington's Pharmaceutical Sciences, 1985, pp. 1518-1541, Gennaro, A. R., ed., Mack Publishing Company, Easton, Pa.), which is incorporated herein in its entirety by reference. Such methods of administration may include, but are not limited to, surface application, oral, or parenteral routes, injection into joints, subcutaneous injection, or other pharmaceutical methods of delivery. Surface application of the oligonucleotide includes topical application to such surfaces as skin, eyes, lungs, nasal or oral passages, ears, rectum, vagina, and the like. Appropriate means for parenteral administration include 5% dextrose, normal saline, Ringer's solution and Ringer's lactate. The oligonucleotide may be stored as a lyophilized powder and reconstituted when needed by addition of an appropriate salt solution.

### (c) Target Nucleic Acid

The nucleic acid that is targeted for silencing can and will vary depending upon the application. The target nucleic acid may be deoxyribonucleic acid (DNA) or ribonucleic acid (RNA). Typically, the target RNA is messenger RNA (mRNA).

In some embodiments, the target nucleic acid may be endogenous to the cell. For example, the endogenous target nucleic acid may be a naturally occurring nucleic acid or a mutated version of a naturally occurring nucleic acid. The aberrant expression (either directly or indirectly) of a naturally occurring nucleic acid may result in a disease state. Examples of suitable disease states include, but are not limited to, genetic disorders, cancers, CNS disorders, cardiovascular disorders, metabolic disorders, inflammatory disorders, autoimmune disorders, and so forth.

In other embodiment, the target nucleic acid may be exogenous to the cell. For example, exogenous nucleic acid may be from a virus (e.g., HIV) or other pathogen (e.g., Plasmodium falciparum) that has infected the cell. In these instances, the antisense region of oligonucleotide typically is complementary to a portion of the target nucleic acid essential to the metabolism, growth, or reproduction of the virus or other pathogen, wherein the inhibition of expression results in partial or full, temporary or permanent alleviation of the effects of the infection. Alternatively, the exogenous nucleic acid may be have been explicitly introduced into the cell, wherein the inhibition of its expression is desired for research purposes.

The oligonucleotide of the invention may silence or reduce the expression of the target nucleic acid by cleavage and degradation of the target nucleic acid, inhibition of translation of the transcript, or a combination thereof. In general, expression of the target nucleic acid may be reduced by at least about 20%. In some embodiments, the expression of the target nucleic acid may be reduced by about 20%, about 30%, about 40%, about 50%, about 60%, about 70%, about 80%, about 85%, about 90%, about 95%, or about 99%. An advantage of the method is that the silencing of unintended target nucleic acids is reduced. The number of off-target nucleic acids that may be affected by a particular oligonucleotide can and will vary depending upon the specific nucleic acids. Preferably, the oligonucleotide of the invention may reduce the expression of an off-target nucleic acid by no more than about 50%. For example, the oligonucleotide may reduce expression of an off-target nucleic acid by about 50%, about 40%, about 30%, about 25%, about 20%, about 15%, about 10%, about 5%, or about 1%.

### (II) Oligonucleotides

Another aspect of the invention encompasses an oligonucleotide. The oligonucleotide comprises a duplex portion comprising a sense region base paired with an antisense region, wherein the antisense region comprises a 5′ phosphate group on the first nucleotide and at least one 2′-5′ internucleotide linkage in the region from the second nucleotide to the eighth nucleotide from the 5′ end. The oligonucleotides of the invention are detailed above in section (I)(a), and may be used in the processes detailed above in section (I).

### DEFINITIONS

To facilitate understanding of the invention, several terms are defined below.

The term “acyl,” as used herein alone or as part of another group, denotes the moiety formed by removal of the hydroxy group from the group COOH of an organic carboxylic acid, e.g., RC(O)—, wherein R is R₁, R₁O—, R₁R₂N—, or R₁S—, R₁ is hydrocarbyl, heterosubstituted hydrocarbyl, or heterocyclo, and R₂ is hydrogen, hydrocarbyl or substituted hydrocarbyl.

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aryl” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

As used herein, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complimentary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between a duplex region may be partial (e.g., 70%), if only some of the base pairs are complimentary. The bases that are not complementary are “mismatched.” Complementarity may also be complete (i.e., 100%), if all the base pairs of the duplex region are complimentary.

The terms “halogen” or “halo” as used herein alone or as part of another group refer to chlorine, bromine, fluorine, and iodine.

The term “heteroatom” means atoms other than carbon and hydrogen.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, sulfur, or a halogen atom. These substituents include halogen, heterocyclo, alkoxy, alkenoxy, aryloxy, hydroxy, protected hydroxy, acyl, acyloxy, nitro, amino, amido, nitro, cyano, ketals, acetals, esters and ethers.

The term “off-target,” as used herein, refers to a nucleic acid that is unintentionally silenced by RNA interference.

The term “target,” as used herein, refers to a nucleic acid that is intentionally silenced by RNA interference.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Example 1

### Modified siRNAs have Reduced Off-Target Activity

A variety of siRNA duplexes with different modifications in the sense and/or antisense strand were tested for their ability to reduce the levels of a specific target mRNA (i.e., mitogen-activated protein kinase 14, MAPK14). Table 1 presents the modifications. Each of the unmodified and the modified siRNAs had a 5′ terminal phosphate on the antisense strand.

TABLE 1
siRNA Modifications.

| siRNA | Description |
|---|---|
| MAPK14-193 | Unmodified |
| 2′-OMe | 2′-OMe in position 2 of antisense strand and |
| | 2′-OMe in position 1 and 2 of sense strand |
| 2′-LNA2-NH₂ | 2′-LNA in position 2 of antisense strand and |
| | amino group with 6-carbon linker at terminal 5′ |
| | phosphate of sense strand |
| 2′-LNA2 | 2′-LNA in position 2 of antisense strand and |
| | 2′-OMe in position 1 and 2 of sense strand |
| 2′-LNA3 | 2′-LNA in position 3 of antisense strand and |
| | 2′-OMe in position 1 and 2 of sense strand |
| 2′-LNA4 | 2′-LNA in position 4 of antisense strand and |
| | 2′-OMe in position 1 and 2 of sense strand |
| 2′-F | 2′-fluoro in position 2 of antisense strand and |
| | 2′-OMe in position 1 and 2 of sense strand |

HeLa cells were transfected with one of the MAPK14 siRNAs or were mock transfected (i.e., transfection reagent only). After a period of incubation the RNA was isolated from the cells and subjected to microarray analysis (i.e., Whole Human Genome Microarray 4×44K platform, Agilent Technologies, Santa Clara, Calif.). The 2′-OMe, 2′-F, and 2′-LNA siRNAS reduced the level of the target transcript (relative to the mock control) (data not shown). To estimate the number of genes showing off-target effects, the microarray data was analyzed using GENESIFTER® microarray analysis software (ViZxlabs, Seattle, Wash.) to search for a pattern in which the unmodified siRNA showed an off-target down regulation of greater than two-fold but was restored to within 10% of the mock control by a particular modification. Pattern searching was conducted with ANOVA tests using a correlation coefficient of 0.98 and a p-cutoff of 0.05. This analysis revealed that the 2′-LNA and 2′-F siRNAs did not reduce the number of off-target knockdowns relative to that provided by the 2′-OMe siRNA (see Table 2).

TABLE 2
Search Pattern for Relative Expression Levels.

| Mock | 1 | 1 | 1 | 1 | 1 | 1 |
|---|---|---|---|---|---|---|
| 193 | <0.5 | <0.5 | <0.5 | <0.5 | <0.5 | <0.5 |
| 2′-OMe | >0.9 | <0.5 | <0.5 | <0.5 | <0.5 | <0.5 |
| 2′-LNA2-NH₂ | <0.5 | >0.9 | <0.5 | <0.5 | <0.5 | <0.5 |
| 2′-LNA2 | <0.5 | <0.5 | <0.5 | >0.9 | <0.5 | <0.5 |
| 2′-LNA3 | <0.5 | <0.5 | <0.5 | <0.5 | >0.9 | <0.5 |
| 2′-LNA4 | <0.5 | <0.5 | <0.5 | <0.5 | <0.5 | >0.9 |
| 2′-F | <0.5 | <0.5 | >0.9 | <0.5 | <0.5 | <0.5 |
| # | 262 | 104 | 21 | 33 | 27 | 17 |

# Number of genes showing reduced off-target effects

### Example 2

### Off-Target Activity of Additional Modified siRNAs

A MAPK14 siRNA was designed in which the antisense strand had a terminal 5′ phosphate and a 2′-5′ phosphodiester linkage between the nucleotides at positions 2 and 3, and the sense strand had a 2-OMe group on each of the nucleotides at positions 1 and 2. The effectiveness of this 2′-5′-linked siRNA to specifically and selectively knockdown a target (MAPK14) was compared to the unmodified MAPK14 siRNA with a 5′ terminal phosphate on the antisense strand (i.e., 193) or MAPK14 siRNAs having a 2′-OMe, 2′-methoxyethoxy, or 2′ allyl group at position 2 of the antisense strand. All of these designs had a 5′ terminal phosphate on the antisense strand and a 2′-OMe group on each of the nucleotides at positions 1 and 2 of the sense strand. Each siRNA was transfected into HeLa cells at a concentration of 33 nM. The expression levels of the target nucleic acid (MAPK14) and three off-target nucleic acids with seed regions that matched the siRNA seed region were evaluated using the QUANTIGENE® system (Sigma-Aldrich). The off-targets were ANKFY1 (i.e., ankyrin repeat FYVE domain-containing 1), MARK2 (i.e., microtubule affinity-regulating kinase 2), and CTNNB1 (i.e., catenin, beta 1). A negative control nucleic acid, CSNK1A1 (i.e., casein kinase 1, alpha 1), which lacked a matching seed region was also included in the experiment.

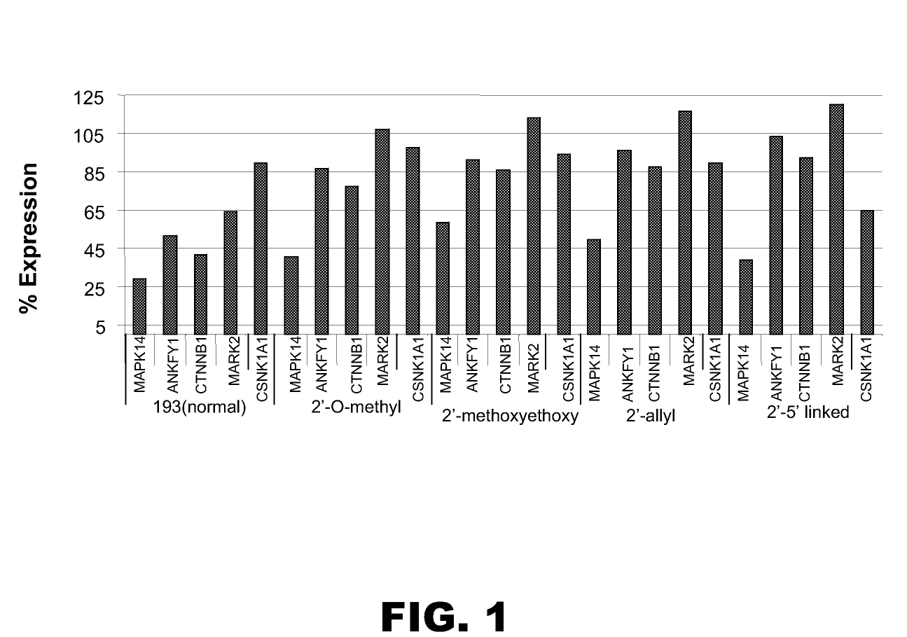

The unmodified, 2′-OMe, and 2′-5′-linked siRNAs were most effective in silencing the target (FIG. 1). Each siRNA reduced MAPK14 expression by approximately 70%. The off-target effects of the 2′-5′-linked siRNA, however, were reduced relative to those of the unmodified and the 2′-OMe siRNAs.

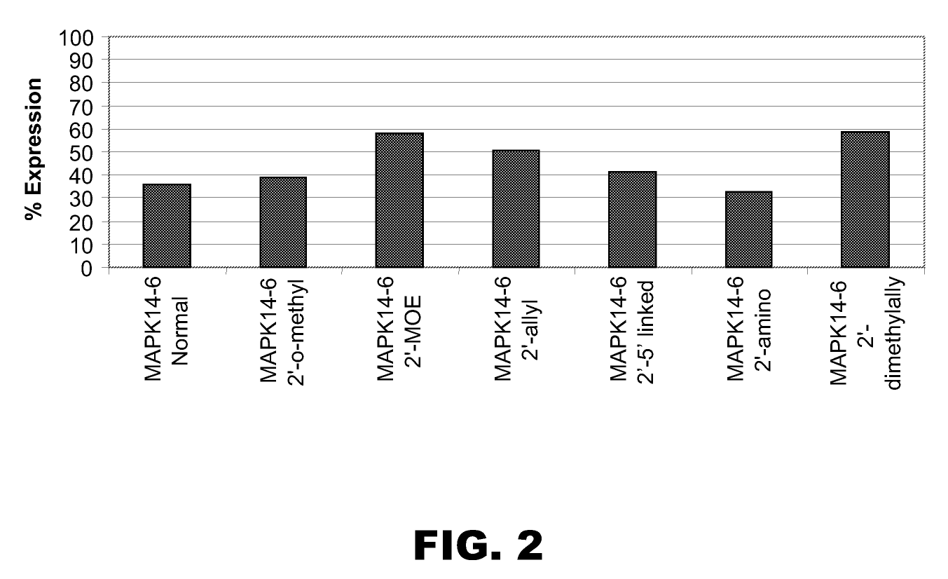

The effects of another MAPK14 siRNA sequence were tested. The MAPK14 siRNA was unmodified (MAPK14-6) with a 5′ terminal phosphate group on the antisense strand, or the second nucleotide in the antisense strand had a 2′-OMe, 2′-methoxyethoxy, 2′-allyl, 2′-amino, 2′-dimethylally, or 2′-5′ linkage modification. All of these chemically modified antisense strand designs had a 5′ terminal phosphate on the antisense strand and a 2′-OMe group on each of the nucleotides at positions 1 and 2 of the sense strand. Specific MAPK14 knockdown using these modified siRNAs was measured using the QUANTIGENE® system. The 2′-OMe siRNA and the 2′-5′-linked siRNA reduced MAPK14 expression by about 65% (FIG. 2). Testing another MAPK14 siRNA sequence ensured that the MAPK14 downstream pathways were controlled for, and the off-target effects were primarily due to siRNA seed interactions with identical seed matches of extraneous transcripts.

### Example 3

### Microarray Analysis of Off-Target Activity

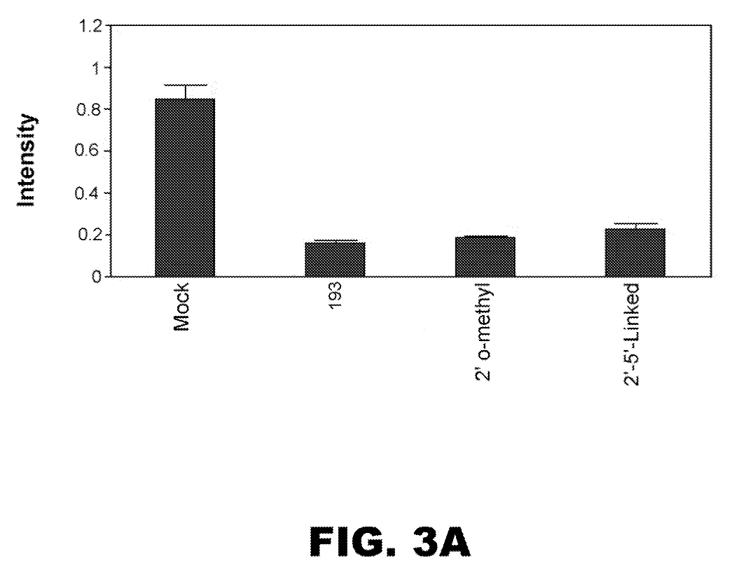

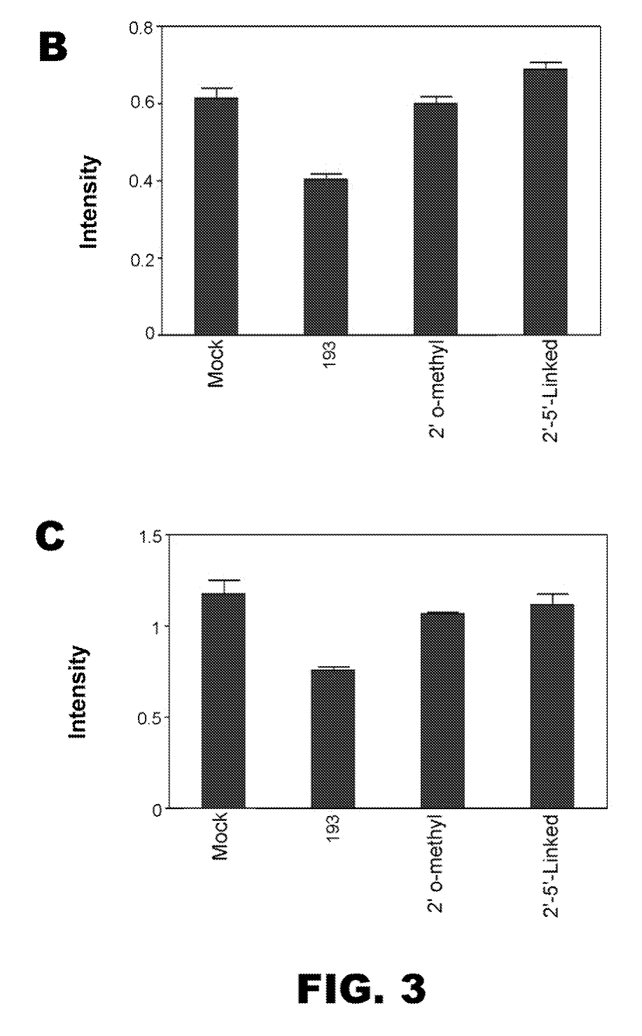

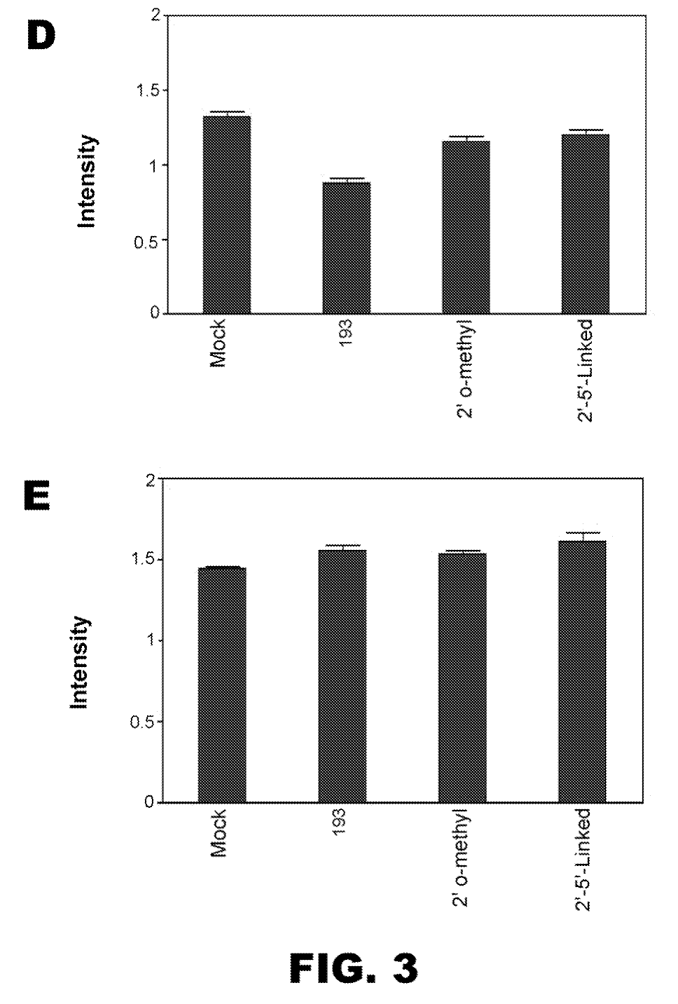

To better quantify the knockdown effects of the unmodified (193), 2′-5′-linked, and 2′-OMe siRNAs described in Example 2, they were subjected to microarray analysis essentially as described in Example 1. GENESIFTER® microarray heat map analysis software was used to measure the intensity levels of the expression signals. Tables 3, 4, 5, 6, and 7 present the data (and FIGS. 3A, 3B, 3C, 3D, and 3E plot the intensities from the heat maps) for the target (MAPK14), the three off-targets (CTNNB1, ANKFY1, MARK2), and the negative control (CSNK1A1), respectively. All three MAPK14 siRNAs reduced expression of MAPK14, but the 2′-5′-linked siRNA had significantly reduced off-target effects relative to the unmodified siRNA and generally less off-target effects than the 2′-OMe siRNA.

TABLE 3
MAPK14 Analysis.

| Condition | Intensity | SEM | SEM/Intensity | Quality |
|---|---|---|---|---|
| Mock | 0.8462 | ±0.0672 | 7.9% | 1.0000 |
| 193 | 0.1627 | ±0.0098 | 6.0% | 1.0000 |
| 2′-OMe | 0.1868 | ±0.0036 | 1.9% | 1.0000 |
| 2′-5′-linked | 0.2299 | ±0.0227 | 9.9% | 1.0000 |

TABLE 4
CTNNB1 Analysis.

| Condition | Intensity | SEM | SEM/Intensity | Quality |
|---|---|---|---|---|
| Mock | 0.6146 | ±0.0259 | 4.2% | 1.0000 |
| 193 | 0.4064 | ±0.0127 | 3.1% | 1.0000 |
| 2′-OMe | 0.5986 | ±0.0191 | 3.2% | 1.0000 |
| 2′-5′-linked | 0.6875 | ±0.0186 | 2.7% | 1.0000 |

TABLE 5
ANKGY1 Analysis.

| Condition | Intensity | SEM | SEM/Intensity | Quality |
|---|---|---|---|---|
| Mock | 1.1750 | ±0.0711 | 6.1% | 1.0000 |
| 193 | 0.7582 | ±0.0191 | 2.5 | 1.0000 |
| 2′-OMe | 1.0706 | ±0.0016 | 0.1 | 1.0000 |
| 2′-5′-linked | 1.1189 | ±0.0552 | 4.9 | 1.0000 |

TABLE 6
MARK2 Analysis.

| Condition | Intensity | SEM | SEM/Intensity | Quality |
|---|---|---|---|---|
| Mock | 1.3215 | ±0.0317 | 2.4% | 1.0000 |
| 193 | 0.8725 | ±0.0335 | 3.8% | 1.0000 |
| 2′-OMe | 1.11557 | ±0.0384 | 3.3% | 1.0000 |
| 2′-5′-linked | 1.1980 | ±0.0304 | 2.5% | 1.0000 |

TABLE 7
CSNK1A1 Analysis.

| Condition | Intensity | SEM | SEM/Intensity | Quality |
|---|---|---|---|---|
| Mock | 1.4491 | ±0.0183 | 1.3% | 1.0000 |
| 193 | 1.5609 | ±0.0287 | 1.8% | 1.0000 |
| 2′-OMe | 1.5351 | ±0.0191 | 1.2% | 1.0000 |
| 2′-5′-linked | 1.6157 | ±0.0481 | 3.0% | 1.0000 |

### Example 4

### Global siRNA Off-Target Reduction

To determine whether 2′-5′-linked siRNAs had reduced off-targeting effects relative to 2′-OMe siRNAs, a whole genome microarray was performed essentially as detailed in Example 1. Both MAPK14 siRNA sequences were tested. The MAPK14 2′-5′-linked siRNAs had a statistically significant reduction in the total number of off-target effects as compared to the MAPK14 2′-OMe siRNAs.

The microarray data were analyzed with the GENESIFTER® microarray analysis software. The pattern searching was conducted with ANOVA tests. Three different off-target knockdown levels (intensity levels compared to mock samples) for the unmodified siRNA samples were analyzed. These intensity level cut offs were set at <0.2, <0.25 and <0.3 with respect to the mock samples, whose level of intensity was set at one. For example, the <0.2 intensity level was 5-fold lower than the mock samples. Unmodified siRNA off-targets were considered reduced by siRNA chemical modification if the intensity level of the particular off-target was brought to within 20% or 10% the intensity level of the mock samples. Reduced potential off-targets where evaluated at intensity level thresholds of >0.67, >0.75, >0.8, and >0.9 for the chemically modified siRNAs. For example, the 0.67 intensity level threshold signified a level that is 1.5 fold from the level of the mock samples.

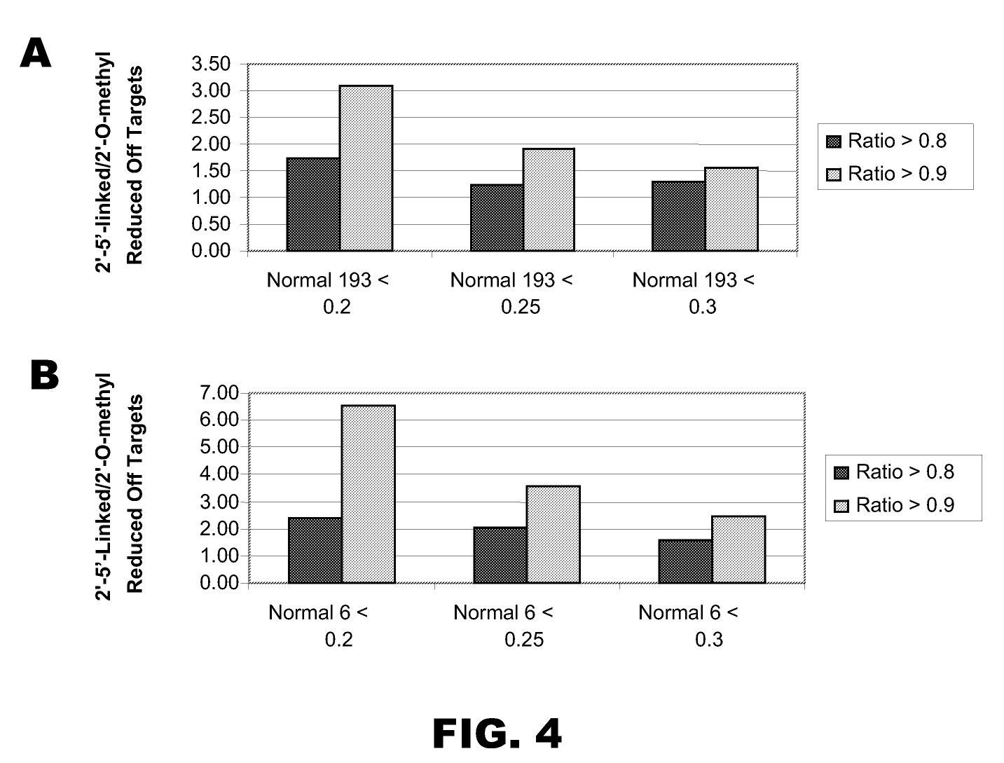

FIGS. 4A and 4B plot the ratio of 2′-5′-linked/2′-OMe siRNA off-target reduction for the two different MAPK14 siRNA sequences. The MAPK14-193 siRNA sequence showed a 3-fold reduction of the number of off-targets by the 2′-5′-linked siRNA with respect to the 2′-OMe siRNA for off-targets that were severely affected by the unmodified siRNA (i.e., at intensity levels below 0.2 when compared with the mock samples) (FIG. 4A). The MAPK14-6 siRNA sequence design, under the same testing and analysis conditions, showed greater than six fold reduction of the number of off-target effects by the 2′-5′-linked siRNA with respect to the 2′-OMe siRNA (FIG. 4B).

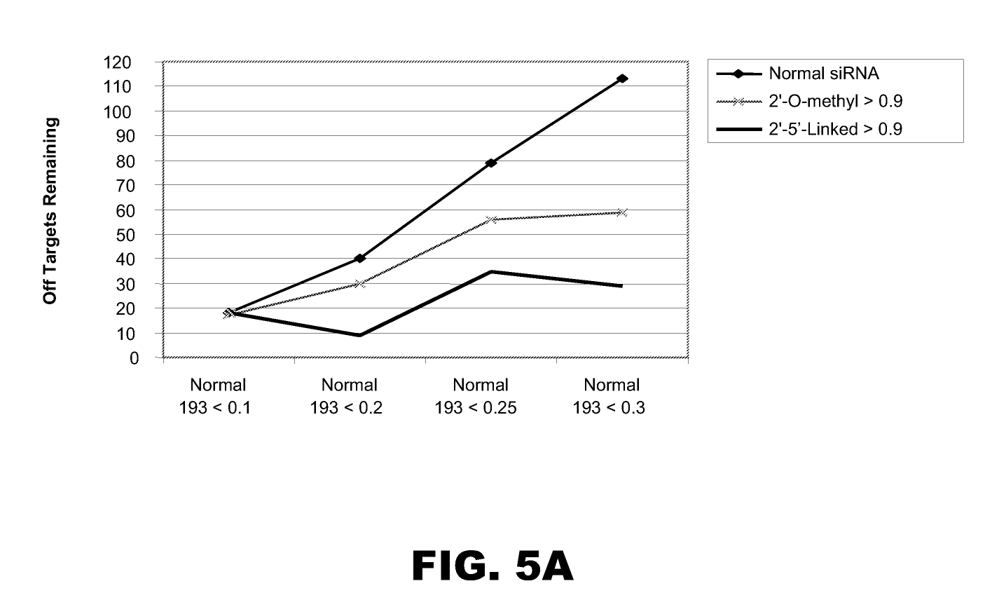

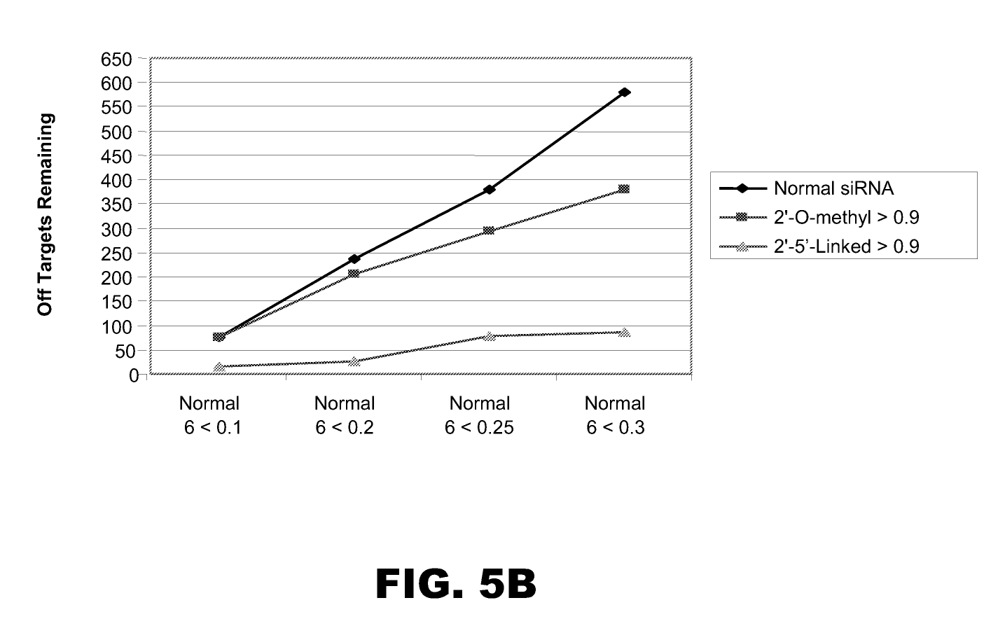

FIGS. 5A and 5B plot the number of off-targets remaining under the different conditions for the two different MAPK14 siRNA sequences. Four different off-target knockdown levels (intensity levels compared to mock samples) for the unmodified siRNA samples were analyzed. These intensity level cut offs were set at <0.1, <0.2, <0.25 and <0.3 when compared to the mock with a set intensity level of one. Unmodified siRNA off-targets were considered reduced by siRNA chemical modification if the intensity level of the particular off-target was brought to within 10% the intensity level of the mock. Under each intensity level cut off, the number of off-targets remaining was significantly reduced by the 2′-5′-linked siRNAs (FIG. 5).

### Example 5

### Global siRNA Off-Target Reduction—Additional Targets

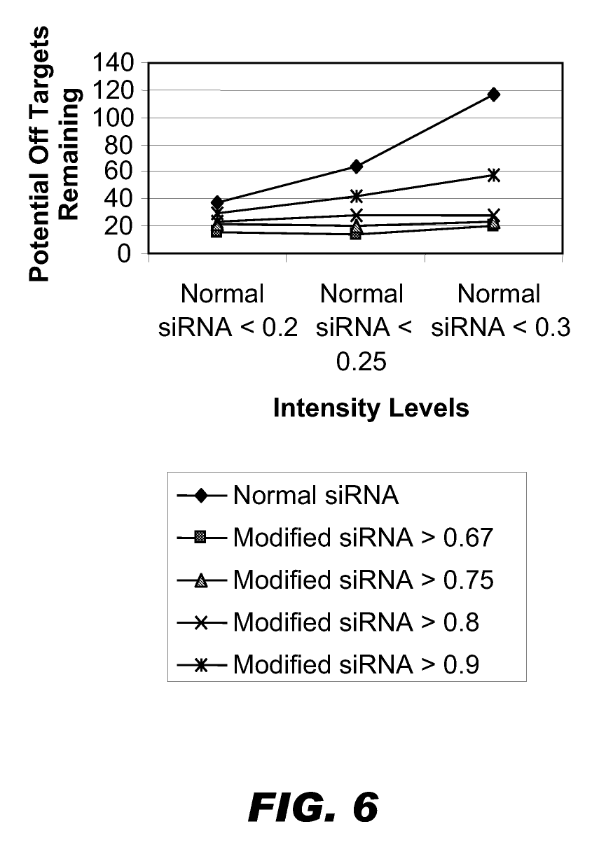

To further assess the reduction of off-target effects of the 2′-5′-linked siRNA, additional target nucleic acid sequences were analyzed. In particular, global siRNA off-target effect reduction was compared between 2′-5′-linked siRNAs and unmodified siRNAs using Agilent Whole Genome Microarrays. Four 2′-5′-linked siRNAs were synthesized against PPP2R2A (i.e., protein phosphatase, regulatory subunit 2, alpha isoform) and knockdown was compared with unmodified siRNA. As shown in FIG. 6, the 2′-5-linked siRNAs significantly reduced off-target effects when compared with unmodified siRNA. Similar results were obtained using 2′-5′-linked siRNAs against MLH1 (i.e., mutL homolog 1, colon cancer, nonpolyposis type 2), JAK1 (i.e., Janus kinase 1), and NLN (i.e., neurolysin) (data not shown). For most of the modified siRNAs, fewer potential off-targets were reduced by the 2′-5′ linkage modification at higher intensity level thresholds.

### Example 6

### Lowest Effective siRNA Concentration

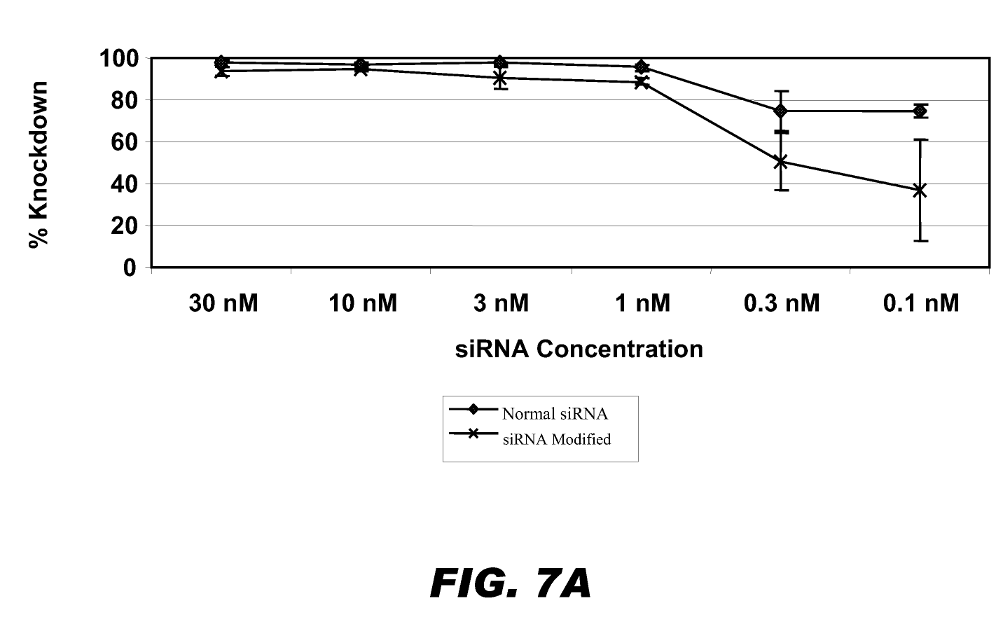

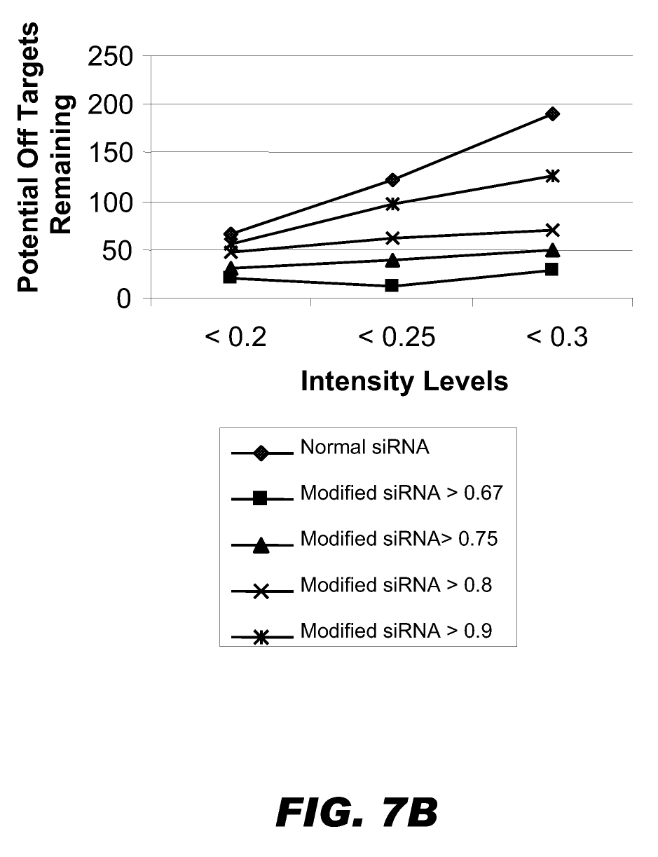

Specific knockdown experiments showed that 1 nM was the lowest effective concentration for both unmodified and 2′-5′-linked siRNAs (see FIG. 7A). After determining 1 nM to be the lowest effective concentration for each type of siRNA, the Whole Human Genome Microarray 4×44K platform from Agilent was used to globally analyze off-target effects from 2′-5′-linked siRNAs designed against TP53 (i.e., tumor protein 53) (see FIG. 7B). Even at these low siRNA concentrations, the global off-target reduction by 2′-5′-linked siRNAs was measurable and statistically significant. Similar experiments were performed using siRNAs against GRB2 (i.e., growth factor receptor-bond protein 2). Again the lowest effective concentration was 1 nM and the 2′-5′-linked siRNAs had a reduced number of potential off-targets remaining.

### Example 7

### Negative Control siRNAs—Off-Target Reduction

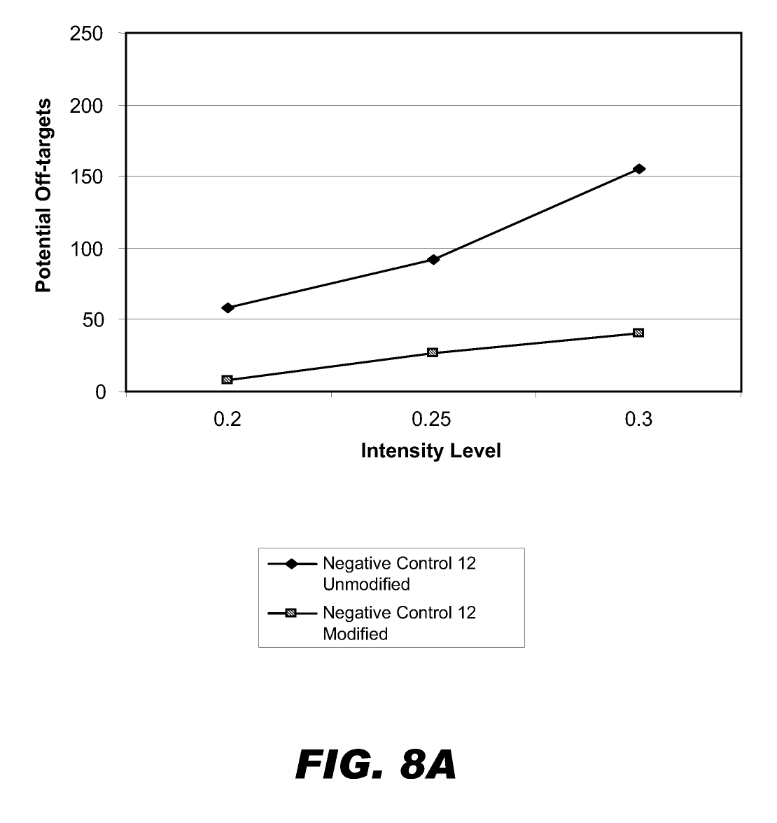

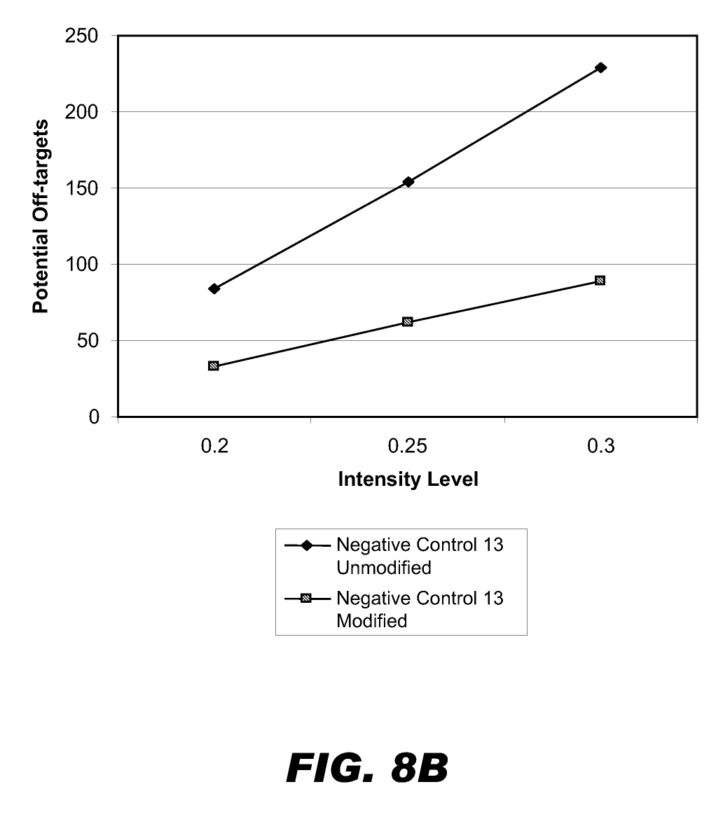

The Whole Human Genome Microarray 4×44K platform from Agilent was used to globally analyze off-target effects from two scrambled negative control siRNAs. Negative control siRNAs 12 and 13 were either unmodified or contained a 2′5′ linkage modification. Both negative control siRNAs (i.e., 12 and 13) were designed to not target any ORF in human, mouse, or rat genomes. As shown in FIG. 8, the 2′-5′-linked negative control siRNAs had significantly fewer off-target effects than the unmodified negative control siRNAs.

### Example 8

### Specific Knockdowns

Specific knockdown was determined by measuring the mRNA transcript levels by quantitative RT-PCR using TaqMan® probes. Several siRNAs were tested at concentrations between 30 nM and 0.1 nM for gene-specific knockdown in HeLa cells. Cell viability, during these knockdown experiments, was also measured using a CellTiter-Glo® kit (Promega, Inc., Madison, Wis.).

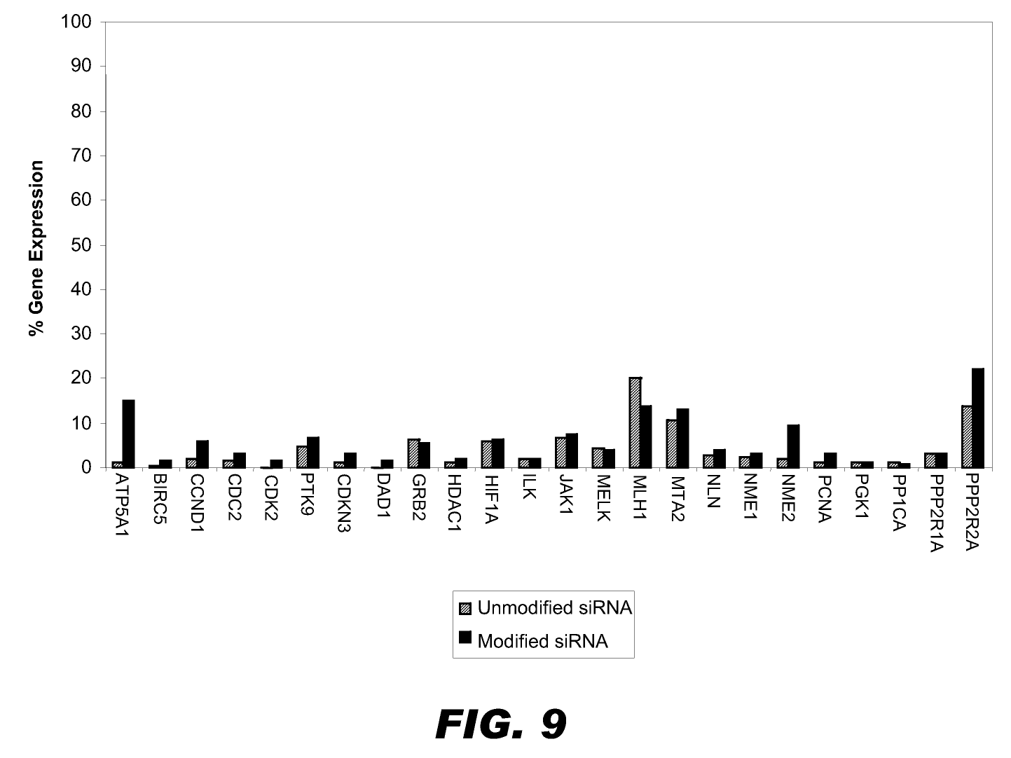

QRT-PCR on 24 genes showed equivalent specific knockdown with either 2′-5′-linked modified siRNA or unmodified siRNA (see FIG. 9). No cell viability problems were detected with either the modified or unmodified siRNAs during these knockdown experiments.

### Example 9

### Global siRNA Off-Target Reduction—Comparison of Different Passenger Strand Designs

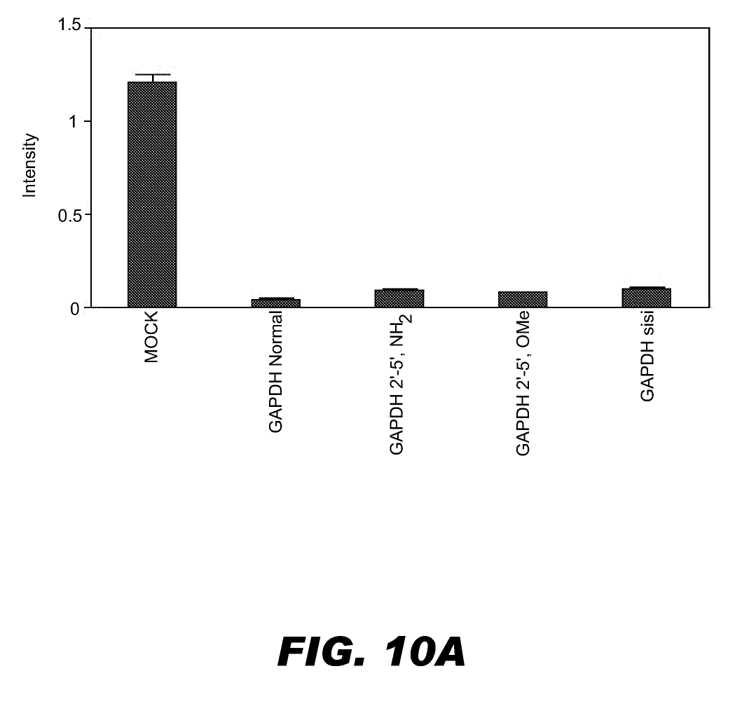

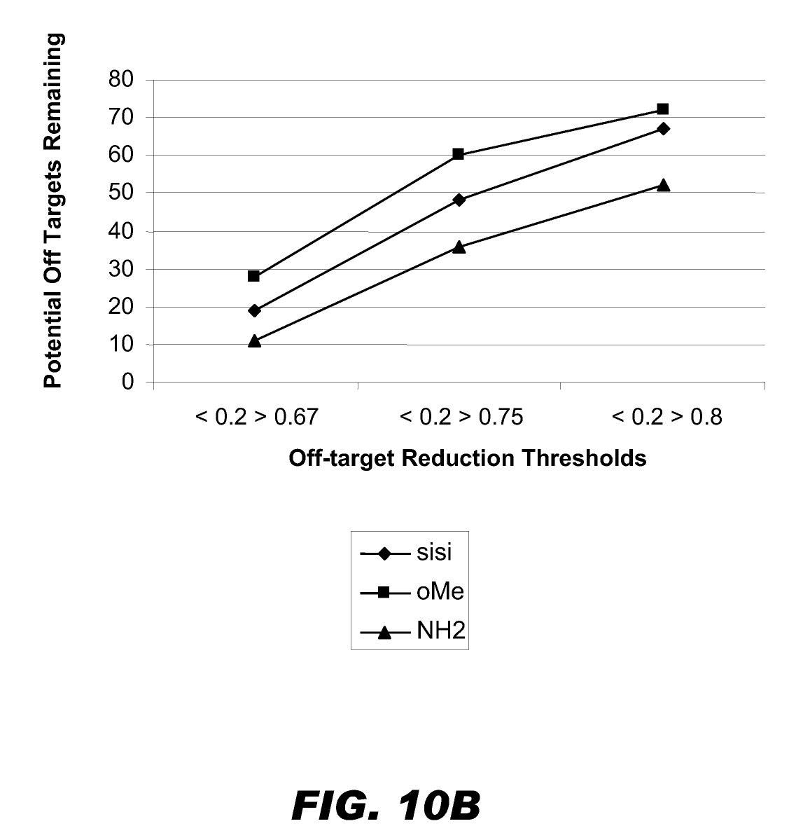

The Whole Human Genome Microarray 4×44K platform from Agilent was used to globally analyze off-target effects from siRNAs targeting GAPDH with different passenger strand designs. The different passenger strands designs were: 2′-5′-linked with a 5′ end amino group; 2′-5′-linked with an O-methyl group; and a small internally segmented interfering RNA (sisiRNA) (Bramsen, et al., Nucleic Acids Res., 2007, 1-12). This analysis revealed that different passenger strand designs significantly reduced passenger strand off-target effects to a similar degree (see FIG. 10).

What is claimed is:

1. A method for specifically silencing the expression of a target nucleic acid in a biological sample, the method comprising contacting the biological sample with an oligonucleotide comprising a duplex portion, the duplex portion comprising a sense region base paired with an antisense region, the antisense region having at least about 70% complementary to the target nucleic acid such that the target nucleic acid is silenced by RNA interference, wherein the antisense region comprises at least one 2′-5′ internucleotide linkage in the region from the second nucleotide to the eighth nucleotide from the 5′ end.

2. The method of claim 1, wherein the silencing of off-target nucleic acids is reduced.

3. The method of claim 2, wherein the internucleotide linkages of the oligonucleotide are selected from the group consisting of phosphorus-containing linkages, non-phosphorus-containing linkages, and combinations thereof.

4. The method of claim 3, wherein the oligonucleotide comprises at least one 3′ overhang, the overhang comprising from one to about six nucleotides.

5. The method of claim 4, wherein the oligonucleotide comprises one antisense strand and at least one sense stand, and the duplex portion comprises from about 15 to about 40 base pairs.

6. The method of claim 4, wherein the oligonucleotide is a single molecule comprising the duplex portion and a loop region, the loop region connecting the duplexed sense and the antisense regions, and the duplex portion comprising from about 15 to about 40 base pairs.

7. The method of claim 5, wherein there is a 5′ phosphate group on the first nucleotide from the 5′ end of the antisense strand.

8. The method of claim 5, wherein there is a 5′ amino group on the first nucleotide from the 5′ end of the sense strand or strands.

9. The method of claim 5, wherein the oligonucleotide further comprises a 2′ substituent on at least one nucleotide in the sense region, the 2′ substituent being selected from the group consisting of hydrogen, halogen, —R, —NHR, —NRR¹, —SR, and —OR, wherein R and R¹ are independently selected from the group consisting of hydrogen, hydrocarbyl, and substituted hydrocarbyl.

10. The method of claim 5, wherein there is at least one 2′-5′ internucleotide linkage in the sense strand.

11. The method of claim 10, wherein at least one of the 2′-5′ linked nucleotides also comprises a 3′ substituent selected from the group consisting of hydrogen, halogen, —R, —NHR, —NRR¹, —SR, and —OR, wherein R and R¹ are independently selected from the group consisting of hydrogen, hydrocarbyl, and substituted hydrocarbyl.

12. The method of claim 5, wherein the oligonucleotide comprises one sense strand, and the duplex portion comprises from about 17 to about 25 base pairs.

13. The method of claim 12, wherein the 2′-5′ linkage is between the second and third nucleotides from the 5′ end of the antisense strand.

14. The method of claim 13, wherein there is a 5′ phosphate group on the first nucleotide from the 5′ end of the antisense strand, and there is a 5′ amino group on the first nucleotide from the 5′ end of the sense strand.

15. The method of claim 13, wherein there is a 5′ phosphate group on the first nucleotide from the 5′ end of the antisense strand, and there a 2′-O-methyl group on each of the first and second nucleotides from the 5′ end of the sense strand.

16. The method of claim 13, wherein there is a 5′ phosphate group on the first nucleotide from the 5′ end of the antisense strand, there is a 2′-O-methyl group on the first nucleotide from the 5′ end of the sense strand, and there is a second 2′-5′ linkage between the second and third nucleotides from the 5′ end of the sense strand.

17. The method of claim 1, wherein the biological sample is a cell or an extract of a cell.

18. The method of claim 17, wherein the cell is disposed in a human or an animal.

19. An oligonucleotide, the oligonucleotide comprising a duplex portion, the duplex portion comprising a sense region base paired with an antisense region, and the antisense region comprising a 5′ phosphate group on the first nucleotide and at least one 2′-5′ internucleotide linkage in the region from the second nucleotide to the eighth nucleotide from the 5′ end.

20. The oligonucleotide of claim 19, wherein the internucleotide linkages of the oligonucleotide are selected from the group consisting of phosphorus-containing linkages, non-phosphorus-containing linkages, and combinations thereof.

21. The oligonucleotide of claim 20, wherein the oligonucleotide comprises at least one 3′ overhang, the overhang comprising from one to about six nucleotides.

22. The oligonucleotide of claim 21, wherein the oligonucleotide comprises one antisense strand and at least one sense stand, and the duplex portion comprises from about 15 to about 40 base pairs.

23. The oligonucleotide of claim 21, wherein the oligonucleotide is a single molecule comprising the duplex portion and a loop region, the loop region connecting the duplexed sense and the antisense regions, and the duplex portion comprising from about 15 to about 40 base pairs.

24. The oligonucleotide of claim 22, further comprising a 5′ amino group on the first nucleotide at the 5′ end of the sense strand.

25. The oligonucleotide of claim 22, further comprising a 2′ substituent on at least one nucleotide in the sense region, the 2′ substituent being selected from the group consisting of hydrogen, halogen, —R, —NHR, —NRR¹, —SR, and —OR, wherein R and R¹ are independently selected from the group consisting of hydrogen, hydrocarbyl, and substituted hydrocarbyl.

26. The oligonucleotide of claim 22, further comprising at least one 2′-5′ internucleotide linkage in the sense strand.

27. The oligonucleotide of claim 26, wherein at least one of the 2′-5′ linked nucleotides also comprises a 3′ substituent selected from the group consisting of hydrogen, halogen, —R, —NHR, —NRR¹, —SR, and —OR, wherein R and R¹ are independently selected from the group consisting of hydrogen, hydrocarbyl, and substituted hydrocarbyl.

28. The oligonucleotide of claim 22, wherein the oligonucleotide comprises one sense strand, and the duplex portion comprises from about 17 to about 25 base pairs.

29. The oligonucleotide of claim 28, wherein the at least one 2′-5′ linkage is between the second and third nucleotides from the 5′ end of the antisense strand.

30. The oligonucleotide of claim 29, further comprising a 5′ amino group on the first nucleotide at the 5′ end of the sense strand or stands.

31. The oligonucleotide of claim 29, further comprising a 2′-O-methyl group on each of the first and second nucleotides at the 5′ end of the sense strand.

32. The oligonucleotide of claim 29, further comprising a 2′-O-methyl group on the first nucleotide at the 5′ end of the sense strand, and a second 2′-5′ linkage between the second and third nucleotides from the 5′ end of the sense strand.
